(19) United States
(12) Patent Application Publication
(10) Pub. No.: US 2024/0408072 A1
(43) Pub. Date: Dec. 12, 2024

(54) COMPOSITION FOR PREVENTING OR TREATING GRAVES' DISEASE COMPRISING COMPOUND CONTAINING AN IMIDAZOPYRIDINE STRUCTURE AS ACTIVE INGREDIENT

(71) Applicant: ESGELBIO INC., Seoul (KR)

(72) Inventor: Cheol Won YUN, Seoul (KR)

(73) Assignee: ESGELBIO INC., Seoul (KR)

(21) Appl. No.: 18/704,068

(22) PCT Filed: Oct. 20, 2022
(86) PCT No.: PCT/KR2022/015988

§ 371 Date: Apr. 24, 2024

(30) Foreign Application Priority Data
Oct. 27, 2021 (KR) 10-2021-0144988
Oct. 19, 2022 (KR) 10-2022-0134912

(51) Int. Cl. A61K 31/444; A61K 31/437; A61P 5/16; A61P 37/06

(52) U.S. Cl. CPC A61K 31/444; A61K 31/437; A61P 5/16; A61P 37/06

(57) ABSTRACT

The present invention relates to a composition for preventing or treating Graves' disease comprising a compound containing an imidazopyridine structure as an active ingredient. Specifically, the present invention relates to a pharmaceutical composition for preventing or treating Graves' disease comprising a compound containing an imidazopyridine structure as an active ingredient, a health functional food for preventing or ameliorating Graves' disease, and a method for treating Graves' disease comprising a step of administering a compound containing an imidazopyridine structure to an animal suffering from Graves' disease.

### TECHNICAL FIELD

The present disclosure relates to a composition including an imidazopyridine scaffold-bearing compound as an active ingredient for preventing or treating Graves' disease.

### BACKGROUND ART

Graves' disease (GD), which is an autoimmune disorder resulting in hyperthyroidism, is characterized by the overproduction of thyroid hormones due to the activation of thyroid receptors by thyroid-stimulating immunoglobulins.

The exact cause of Graves' disease is not yet known, but it is believed to involve a combination of genetic and environmental factors. There is a possibility that individuals with a family member suffering from this disease may also be affected. In the case of twins, if one identical twin is affected, there is a 30% chance that the other twin will also have the disease. Graves' disease can be triggered by stress, infection, or at birth, and is commonly seen in patients with other autoimmune disorders such Type 1 as diabetes and rheumatoid arthritis.

Furthermore, Graves' disease originates from an antibody known as thyroid-stimulating immunoglobulin (TSI), which has a similar effect to thyroid-stimulating hormone. These antibodies induce the thyroid gland to produce excessive thyroid hormone.

Current diagnostic methods for Graves' disease are based on blood tests and symptoms assessed through radioactive iodine uptake, while treatment options include radioactive iodine therapy, medication, and thyroid surgery.

Immediately performing surgery on patients with hyperthyroidism may be dangerous. Thus, antithyroid drugs administered before thyroidectomy are to induce hypothyroidism, and the surgery is carried out afterwards. Antithyroid medication needs to be taken for 6 months to 2 years to be effective, and discontinuing the medication can lead to a relapse of hyperthyroidism, with a relapse risk of about 40-50%. Lifelong treatment with antithyroid drugs may also lead to serious side effects such as agranulocytosis and liver disease, potentially causing a critical decrease in white blood cell count.

Additionally, radioactive iodine therapy involves the injection of radioactive iodine, which accumulates in the thyroid gland and emits radiation in the form of beta and gamma rays. Approximately 90% of the radiation emitted is due to beta (electron) particles. The most common method of iodine therapy involves administering a specific amount based on scintigraphy or radiodiagnostic imaging over 24 hours, targeting 1 g of thyroid tissue for microsclerotherapy. Patients undergoing this treatment must regularly perform thyroid blood tests before significant hypothyroidism symptoms appear.

However, the downside of radioactive iodine therapy is the high incidence of induced hyperthyroidism (up to 80%), and the inconvenience of daily thyroid hormone supplementation. Radioactive iodine is used slowly (over several months) to destroy the thyroid gland, and not all patients with Graves' disease-related hyperthyroidism are treated with radioactive iodine.

Another treatment, surgical resection, has the advantage of being an immediate intervention but comes with risks such as recurrent laryngeal nerve damage, hypoparathyroidism (due to the removal of the parathyroid glands), hematoma (which can be life-threatening if it compresses the trachea), recurrence after treatment, and the potential for infection or scarring.

Therefore, there is a need for the development of new therapeutic agents for Graves' disease that minimize other side effects in the body while providing excellent therapeutic effects.

### DISCLOSURE OF INVENTION

### Technical Problem

Leading to the present disclosure, intensive and thorough research conducted by the present inventors resulted in the finding that compounds containing an imidazopyridine scaffold can be used as new therapeutic agents for the effective treatment and prevention of Graves' disease.

Thus, an aspect of the present disclosure aims to provide a pharmaceutical composition including an imidazopyridine scaffold-bearing compound as an active ingredient for the prevention or treatment of Graves' disease.

Another aspect of the present disclosure is to provide a health functional food including an imidazopyridine scaffold-bearing compound as an active ingredient for the prevention or alleviation of Graves' disease.

A further aspect of the present disclosure is to provide a method for treating Graves' disease, the method including a step of administering an imidazopyridine scaffold-bearing compound to an animal afflicted with Graves' disease.

### Solution to Problem

Therefore, the present disclosure provides a pharmaceutical composition including an imidazopyridine scaffold-bearing compound as an active ingredient for the prevention or treatment of Graves' disease.

In an embodiment of the present disclosure, the compound may be any one selected from the group consisting of Structural Formulas 1 to 3.

| No. | Chemical Structural Formula |
|---|---|
| 1 | 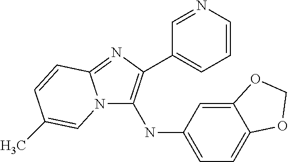 [SMILES: Cc1ccc2nc(-c3cccnc3)c(Nc3ccc4c(c3)OCO4)n2c1] |
| 2 | 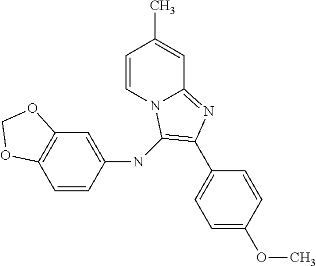 [SMILES: COc1ccc(-c2nc3cc(C)ccn3c2Nc2ccc3c(c2)OCO3)cc1] |
| 3 | 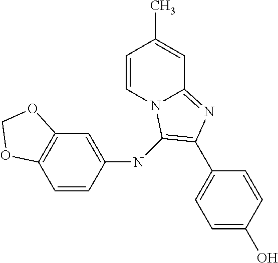 [SMILES: Cc1ccn2c(Nc3ccc4c(c3)OCO4)c(-c3ccc(O)cc3)nc2c1] |

In an embodiment of the present disclosure, the compound may exhibit activities of: inhibiting the expression of thyroid peroxidase (TPO) and thyroglobulin (TG); reducing the size of abnormally enlarged thyroid tissues and cells; and lowering the serum concentration of the thyroid hormone thyroxine.

Additionally, the present disclosure provides a health functional food including an imidazopyridine scaffold-bearing compound as an active ingredient for the prevention or alleviation of Graves' disease.

In an embodiment of the present disclosure, the compound may be any one selected from the group consisting of Structural Formulas 1 to 3.

In an embodiment of the present disclosure, the compound demonstrates activities of inhibiting the expression of thyroid peroxidase (TPO) and thyroglobulin (TG); reducing the size of abnormally enlarged thyroid tissues and cells; and lowering the serum concentration of the thyroid hormone thyroxine.

In another embodiment thereof, the present disclosure provides a method for treating Graves' disease, the method including a step of administering an imidazopyridine scaffold-bearing compound to an animal afflicted with Graves' disease.

In this embodiment, the compound may be any one selected from the group consisting of Structural Formulas 1 to 3.

### Advantageous Effects of Invention

The present disclosure is concerned with composition including an imidazopyridine scaffold-bearing compound as an active ingredient for the prevention or treatment of Graves' disease. The imidazopyridine scaffold-bearing compounds screened in the present disclosure not only exhibit excellent activity in inhibiting the expression of thyroid peroxidase (TPO) and thyroglobulin (TG), but also show significantly superior pharmacological activity compared to currently available drugs. The compounds do not induce toxicity in cellular and animal models, thus confirming the in vivo stability thereof. Hence, the compounds can be promising potential new therapeutic agents for the treatment of Graves' disease.

### BRIEF DESCRIPTION OF DRAWINGS

FIG. 1 illustrates a schematic of the drug screening method for new Graves' disease therapies, as per an embodiment of the present disclosure.

FIG. 2 shows the results of analyzing the inhibitory activity on the expression of TPO (Thyroid Peroxidase) and TG (Thyroglobulin) for the compounds screened in an embodiment of the present disclosure.

FIG. 3 depicts the results of a cytotoxicity analysis conducted on SNU760 Human Thyroid cell lines treated with different concentrations of the candidate drugs of the present disclosure.

FIG. 4 presents the analysis of body weight changes over time in ICR mice animal models after administration of the candidate drugs in an embodiment of the present disclosure.

FIG. 5 is a schematic diagram showing the procedure for the production of a Graves' disease-induced mouse model and the administration process of the candidate drugs in an embodiment of the present disclosure.

FIG. 6 shows the results confirming whether the Graves' disease-induced mice were successfully developed, with part A comparing thyroid tissue size and transparency between the normal control group and the mouse group injected with Ad-TSHR virus, and part B displaying the microscopic observation of thyroid tissue cell size.

FIG. 7 illustrates the comparison of thyroid sizes after excision, following the administration of PTU drug and the imidazopyridine scaffold-bearing compound (NE-2-6) of the present disclosure to the Graves' disease-induced mice in an embodiment of the present disclosure.

FIG. 8 represents the results of measuring the serum thyroxine (Thyroxine) concentration in Graves' disease-induced mice after administration of the PTU drug and the imidazopyridine scaffold-bearing compound (NE-2-6) in an embodiment of the present disclosure.

### BEST MODE FOR CARRYING OUT THE INVENTION

The present disclosure is based on the discovery that imidazopyridine scaffold-bearing compounds can be used as novel drugs for the prevention, treatment, or amelioration of Graves' disease.

Graves' disease is commonly caused by thyroid-stimulating hormone receptor antibodies (TSHRAb) that induce hyperthyroidism, making it the most frequent cause of this condition.

Hyperthyroidism is known to result from the overproduction of thyroid hormone that affects various organs in the body and can increase the risk of morbidity and mortality, such as arrhythmias, heart failure, osteoporosis, and stroke. Like other autoimmune diseases, hyperthyroidism may resolve naturally, but the likelihood is much lower compared to medical treatment, thus necessitating aggressive treatment. However, there are currently no treatments available that can completely cure Graves' disease by inhibiting the production of stimulating TSHRAb; hence, the primary goal of current treatments for Graves' disease is to manage hyperthyroidism and restore normal thyroid function.

In their quest to develop a new treatment for Graves' disease that is effective without causing side effects, the inventors screened approximately 7,000 compounds from the Korea Chemical Bank. As a result, it was experimentally found that imidazopyridine scaffold-bearing compounds have excellent effects of not only exhibiting excellent inhibitory activity against the expression of thyroid peroxidase (TPO) and thyroglobulin (TG) but also reducing the size of abnormally enlarged thyroid tissues and cells and decrease the serum concentration of the thyroid hormone thyroxine. Furthermore, these screened compounds were observed to be free of cytotoxicity, affirming their safety.

In order to develop a novel treatment for Graves' disease, screening processes conducted according to an embodiment of the present disclosure first identified compounds with inhibitory activity against myeloperoxidase (MPO) and subsequently against thyroid peroxidase (TPO).

Myeloperoxidase (MPO) is a heme-containing enzyme found in neutrophils and monocytes and is part of a diverse group of mammalian peroxidases that includes Eosinophil Peroxidase (EPO), Thyroid Peroxidase (TPO), Lactoperoxidase (LPO), and Prostaglandin H Synthase (PGHS). The mature enzyme is a dimer divided equally into halves, each featuring a covalently bound heme that imparts MPO's characteristic green color and exhibits unique spectroscopic properties.

Additionally, systemic levels of MPO are well known as risk factors for various cardiovascular diseases (such as heart failure, acute coronary syndrome, myocardial infarction, stable coronary artery disease, and atherosclerosis-related diseases), and the role of MPO in these pathological conditions is linked not only to oxidative damage caused by its enzymatic products but also to the consumption of nitric oxide, which is a critical regulator of vascular and myocardial cell relaxation. This activity of MPO is also involved in the pathogenesis of Graves' disease.

Furthermore, thyroid peroxidase (TPO) is an enzyme crucial for thyroid hormone production, and the inhibition of TPO, if possible, could potentially prevent or treat Graves' disease.

In an embodiment of the present disclosure, it was found that all the screened candidate drugs with inhibitory activity against MPO and TPO are derivatives bearing the imidazopyridine scaffold.

Experiments were conducted to verify the potential use of these screened imidazopyridine scaffold-bearing compounds as treatments for Graves' disease. To this end, BALB/c female mice were injected with an adenovirus expressing the thyroid stimulating hormone receptor (Ad-TSHR) to induce Graves' disease, and the candidate drugs were administered to these diseased mice to analyze the possibility of alleviation and treatment of Graves' disease.

The results showed that the treatment with the candidate compounds effectively reduced the abnormally enlarged thyroid tissue and cell size, which is a typical symptom of induced Graves' disease, and significantly decreased the elevated concentrations of the thyroid hormone thyroxine (T4) in the serum. Moreover, the therapeutic effects of these candidate drugs on Graves' disease were found to be superior to those of the well-known drug propylthiouracil (PTU).

These findings allowed the inventors to confirm the potential of using the imidazopyridine scaffold-bearing compounds screened in the present disclosure as a treatment for Graves' disease in an animal model.

Therefore, the present disclosure provides a pharmaceutical composition including an imidazopyridine scaffold-bearing compound as an active ingredient for the prevention or treatment of Graves' disease.

| No. | Chemical Structural Formula | Compound Name |
|---|---|---|
| 1 | 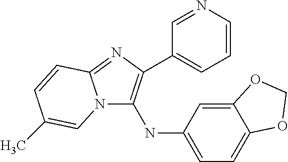 [SMILES: Cc1ccc2nc(-c3cccnc3)c(Nc3ccc4c(c3)OCO4)n2c1] | N-(2H-1,3-BENZODIOXOL-5- YL)-6-METHYL-2-(PYRIDIN-3- YL)IMIDAZO[1,2-A]PYRIDIN-3- AMINE |
| 2 | 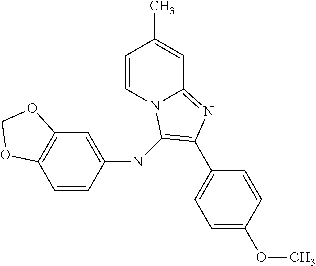 [SMILES: COc1ccc(-c2nc3cc(C)ccn3c2Nc2ccc3c(c2)OCO3)cc1] | N-(2H-1,3-BENZODIOXOL-5- YL)-2-(4-METHOXYPHENYL)-7- METHYLIMIDAZO[1,2- A]PYRIDIN-3-AMINE |
| 3 | 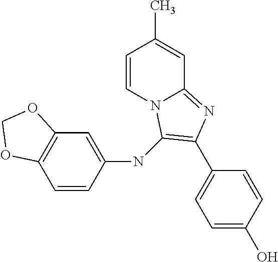 [SMILES: Cc1ccn2c(Nc3ccc4c(c3)OCO4)c(-c3ccc(O)cc3)nc2c1] | 4-[3-(1,3-Benzodioxol-5-ylamino)- 7-methylimidazo[1,2-a]pyridin-2- yl]phenol |

In addition, the imidazopyridine scaffold-bearing compound according to the present disclosure may be used in the form of pharmacologically acceptable salts. The salts may be acid addition salts formed from pharmacologically acceptable free acids. The salts can be derived from a variety of non-toxic inorganic acids including hydrochloric acid, nitric acid, phosphoric acid, sulfuric acid, hydrobromic acid, hydroiodic acid, nitrous acid, or hypophosphorous acid; and from non-toxic organic acids such as aliphatic mono- and dicarboxylates, phenyl-substituted alkanoates, hydroxyalkanoates, and alkanedioates, aromatic acids, and aliphatic and aromatic sulfonic acids. Examples of pharmacologically non-toxic salts include sulfates, pyrosulfates, bisulfates, sulfites, bisulfites, nitrates, phosphates, monohydrogen phosphates, dihydrogen phosphates, metaphosphates, pyrophosphates, chlorides, bromides, iodides, fluorides, acetates, propionates, decanoates, caprylates, acrylates, formates, isobutyrates, caprates, heptanoates, propiolates, oxalates, malonates, succinates, suberates, sebacates, fumarates, maleates, butin-1,4-dioates, hexan-1,6-dioates, benzoates, chlorobenzoates, methylbenzoates, dinitrobenzoates, hydroxybenzoates, methoxybenzoates, phthalates, terephthalates, benzenesulfonates, chlorobenzenesulfonates, toluenesulfonates, xylenesulfonates, phenylacetates, phenylpropionates, phenylbutyrates, citrates, lactates, β-hydroxybutyrates, glycolates, malates, tartrates, methanesulfonates, propanesulfonates, naphthalene-1-sulfonates, naphthalene-2-sulfonates, and mandelates.

Also, the pharmaceutical composition of the present disclosure may further comprise a pharmaceutically acceptable additive, and examples of the pharmaceutically acceptable additive include starch, gelatinized starch, microcrystalline cellulose, lactose, povidone, colloidal silicon dioxide, calcium hydrogen phosphate, lactose, mannitol, taffy, Arabic gum, pregelatinized starch, corn starch, powdery cellulose, hydroxypropylcellulose, Opadry, sodium starch glycolate, Carnauba wax, synthetic aluminum silicate, stearic acid, magnesium stearate, aluminum stearate, calcium stearate, and sucrose.

The pharmaceutically acceptable additive according to the present disclosure may be included at 0.1 to 90 parts by weight with respect to the composition, but with no limitations thereto.

In the present disclosure, the composition may be administered in various oral or parenteral dosage forms at the time of actual clinical administration. When formulated, diluents or excipients such as fillers, extenders, binders, wetting agents, disintegrants, surfactants, etc., may be used. It is preferable to use the materials disclosed in literature (Remington's Pharmaceutical Science, latest edition, Mack Publishing Company, Easton PA.) as a suitable formulation known in the art. Carriers, excipients, and diluents that may be contained in the composition are exemplified by lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, mineral oil, etc.

The solid preparations for oral administration may include tablets, pills, powders, granules, capsules, and the like, and such solid preparations may be prepared by mixing the composition with at least one excipient, such as starch, calcium carbonate, sucrose, lactose, gelatin, etc. In addition to simple excipients, lubricants such as magnesium stearate and talc may also be used. Further, liquid preparations for oral administration may include suspensions, solvents, emulsions, syrups, and the like, and may further include various excipients other than water and liquid paraffin as commonly used simple diluents, for example, wetting agents, sweeteners, fragrances, preservatives, etc.

Formulations for parenteral administration may include sterilized aqueous non-aqueous solutions, solutions, suspensions, emulsions, lyophilized formulations, and suppositories.

The non-aqueous solvents or suspensions may include propylene glycol, polyethylene glycol, vegetable oil such as olive oil, injectable ester such as ethyl oleate, etc. A basic material of the suppository agent may include Witepsol, macrogol, tween 61, cacao butter, laurin butter and glycerol-gelatin. The parenteral administration may be conducted topically or by intraperitoneal, rectal, subcutaneous, intravenous, intramuscular, or intrathoracic injection.

The pharmaceutical composition of the present disclosure may be administered in a pharmaceutically effective amount.

The term “pharmaceutically effective amount” used herein refers to an amount sufficient for the treatment of a disease at a reasonable benefit/risk ratio applicable to a medical treatment, and the level of the effective dose may be determined according to factors including type of a subject, severity of illness, age, sex, kind of infecting viruses, drug activity, drug sensitivity of a subject, administration time, administration route and dissolution rate, treatment duration, and co-administered drugs, and other factors well known in the medical field.

The pharmaceutical composition of the present disclosure may be administered as an individual therapeutic agent or in combination with other therapeutic agent(s), and also sequentially or simultaneously with the conventional therapeutic agent(s). The administration may be conducted once or many times. Additionally, it is important that an amount which can achieve the maximum effect with the least amount without any side effects be administered in consideration of all the factors described above, and can be determined by a person skilled in the art.

The term “administration”, as used herein, means providing a given composition of the present disclosure to a subject by any suitable method.

In the present disclosure, the composition may be administered to an individual through various routes. All modes of administration are contemplated, for example, orally, rectally or intravenously, intramuscularly or subcutaneously.

In the present disclosure, the term “prevention” refers to all actions that suppress Graves' disease or delay its onset by administering a composition.

In the present disclosure, “alleviation” refers to any action that reduces or beneficially changes the symptoms of Graves' disease by administering the composition.

In the present invention, “treatment” refers to any act of curing Graves' disease by administering a composition.

Additionally, the present disclosure provides a method for treating Graves' disease, the method including a step of administering an imidazopyridine scaffold-bearing compound to an animal in which Graves' disease has been induced.

The term “animal” in this context includes all mammals, encompassing humans.

Furthermore, the present disclosure provides a health functional food including an imidazopyridine scaffold-bearing compound as an active ingredient for the prevention or alleviation of Graves' disease.

In an embodiment of the present disclosure, the compound can be any one selected from a group consisting of Structural Formulas 1 to 3.

As used herein, the term “food” refers to a natural or artificial product comprising at least one nutrient, and more preferably, refers to a product which became edible through certain processing, usually encompassing all of food, food additives, health functional food and functional beverages.

The food that may comprise the composition for the prevention or alleviation of Graves' disease according to the present disclosure as an additive may include, for example, different types of food, beverages, chewing gum, tea, vitamin complex, or functional food. In addition, the food of the present disclosure includes special nutritional food (e.g., modified milk, infant/baby food), processed meat products, fish meat products, tofu, muk, noodles (e.g., ramen, Asian noodles), bakery products, health supplement food, seasoning products (e.g., soy sauce, soybean paste, red pepper paste, mixed paste), sauces, confectionery (e.g., snack foods), candies, chocolates, chewing gums, ice-creams, milk products (e.g., fermented milk, cheese), other processed food, Kim-chi, salted food (e.g., different types of Kim-chi, pickled food), beverages (e.g., fruit juice, vegetable juice, soy milk, fermented beverages), and natural seasonings (e.g., broth powder for ramen), but not limited thereto. The food, beverages or food additives can be prepared in conventional manners.

The term “functional food”, as used herein, refers to a group of food to which value is added so as for the function thereof to be exerted and expressed for the predetermined purpose by using physical, biochemical or bioengineering techniques thereto, or a processed food designed so as for the in-vivo adjustment functions of the relevant food composition such as rhythm adjustment in prophylaxis, prevention of disease and recovery from disease to be sufficiently expressed. Such functional food may comprise food additives which are sitologically acceptable, and may additionally comprise suitable carriers, excipients and diluents, which are commonly used in the manufacturing thereof.

The term “beverages”, as used herein, collectively refer to the drink products to relieve thirst or to enjoy the taste. There is no particular limitation thereto, except that, as essential ingredients of the indicated ratio, a composition for prevention and alleviation of Graves' disease should be comprised in the beverages, and various flavoring agents or natural carbohydrates may be contained therein as additional ingredients like in common beverages.

In addition to the foregoing, the food containing the composition for the prevention and alleviation of Graves' disease symptoms according to the present disclosure may various nutrients, vitamins, minerals (electrolyte), flavoring agents such as synthetic flavoring agents and natural flavoring agents, coloring agents and fillers (cheese, chocolate, etc.), pectic acid and salts thereof, alginic acid and salts thereof, organic acids, protective colloidal thickening agents, pH controlling agents, stabilizing agents, preservatives, glycerin, alcohol, carbonizing agents as used in carbonated beverages and the like, and each of the above ingredients may be used alone or in combination with each other.

In the food containing the composition for the prevention and alleviation of Graves' disease symptoms according to the present disclosure, the composition of the present disclosure may be comprised in an amount of 0.001% by weight to 90% by weight, and preferably 0.1% by weight to 40% by weight, based on the total weight of the food; in the case of beverages, it may be comprised at an amount of 0.001 g to 2 g, and preferably 0.01 g to 1 g, based on 100 ml. For long-term intake for the purpose of health and hygiene or for the purpose of health control, however, the amount may be below the above-mentioned range; and since the effective ingredients have no problem in terms of safety profile, they can be used at an amount above the range and they are not limited to the amount range mentioned above.

### MODE FOR CARRYING OUT THE INVENTION

A better understanding of the present disclosure may be obtained through the following Examples, which are set forth to illustrate, but are not to be construed to limit, the scope of the disclosure.

### Example 1

Screening of Compounds with Therapeutic Activity for Graves' Disease
<1-1> Screening of Compounds with Inhibitory Activity Against Myeloperoxidase (MPO)

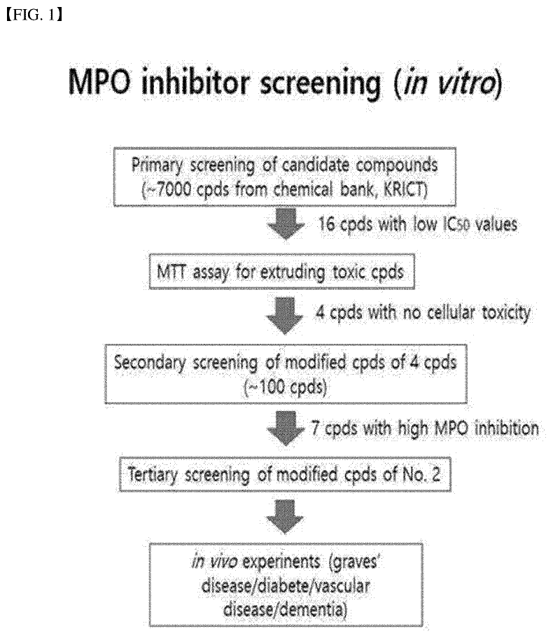

To discover new therapeutics for the treatment of Graves' disease, the inventors screened compounds for inhibition activity against myeloperoxidase (MPO). The screening process is schematically represented in FIG. 1. Use was made of approximately 7,000 compounds for the screening.

The screening of compounds for MPO inhibition activity was conducted in three stages. The inhibitory activity of each compound against MPO was assessed by measuring the IC₅₀ values, which represent the concentration of the compound required to inhibit 50% of the MPO activity. The results of the compounds selected through the second and third stages of screening are displayed in the table below:

TABLE 1
Chemical Library 2^{nd} Screening and Hit IC₅₀ Screening Result
Chemical Library 2nd Screening [symbol] Hit IC₅₀

[symbol]

| | | HL-60 MPO | | MTT Assay |
|---|---|---|---|---|
| | | Inhibition (%) | HL-60 MPO | (Relative death |
| | | (Treat to | Inhibition (%) | rate at 50 uM, |
| | IC₅₀ (uM) | cell lysate) | (treat to media) | HEK293T) |
| AZD3241 | 0.021 | 98 | 0 | 6 |
| (PC) | | | | |
| NE-1 | 0.6 | 70 | 20 | 45 |
| NE-2 | 1.53 | 95 | 0 | 0 |
| NE-3 | 1.98 | 98 | 5 | 0 |
| NE-4 | 1.52 | 98 | 0 | 5 |
| NE-5 | 2 | 71 | 15 | 5 |
| NE-6 | 1.6 | 98 | 0 | 5 |
| NE-7 | 1.24 | 95 | 0 | 5 |
| NE-8 | 0.32 | 94 | 0 | 5 |
| NE-9 | 1.9 | 90 | 0 | 7 |
| NE-10 | 1.97 | 90 | 0 | 3 |
| NE-11 | 1.84 | 90 | 0 | 3 |
| NE-12 | 0.93 | 90 | 0 | 15 |
| NE-13 | 1.02 | 95 | 0 | 5 |
| NE-14 | 1.59 | 98 | 0 | 5 |
| NE-15 | 1.91 | 90 | 0 | 7 |
| NE-16 | 1.64 | 80 | 25 | 35 |

TABLE 2
Chemical Library 3^{rd} Screening and Hit IC₅₀ Screening Result
Chemical Library 3nd Screening [symbol] Hit IC₅₀

[symbol]

| | | HL-60 MPO | HL-60 MPO | |
|---|---|---|---|---|
| | | Inhibition (%) | Inhibition (%) | |
| | | (25 uM of | (25 uM of | MTT Assay |
| | IC₅₀ (uM) | Drugs are | Drugs are | (Relative death |
| | (Human | treated to | treated | rate at 50 uM, |
| No. | MPO) | lysate) | to media) | HEK293T) |
| AZD3241 | 0.021 | 98 | 0 | 5 |
| (PC) | | | | |
| NE-2 | 1.53 | 95 | 0 | 0 |
| NE-4 | 1.52 | 98 | 0 | 5 |
| NE-6 | 1.6 | 98 | 0 | 5 |
| NE-7 | 1.24 | 95 | 0 | 5 |
| NE-8 | 0.32 | 94 | 0 | 5 |
| NE-13 | 1.02 | 95 | 0 | 5 |
| NE-14 | 1.59 | 98 | 0 | 5 |

<1-2> Screening of Compounds with Thyroid Peroxidase (TPO) Inhibition Activity

Following the successful identification of compounds with potent myeloperoxidase (MPO) inhibition activity from the second and third rounds of screening, these compounds were further tested for their ability to inhibit thyroid peroxidase (TPO). The aim was to select compounds that exhibited both and inhibition excellent MPO TPO activities. The selected compounds are presented in Table 3 below, and upon analysis of their structures, it was confirmed that these compounds included the imidazopyridine scaffold. Propylthiouracil (PTU), a conventional medication available on the market, was used as a positive control in this screening process.

TABLE 3

| | Molecular | | Molecular | IC50 (uM) | IC50 (uM) | IC50 (uM) |
|---|---|---|---|---|---|---|
| Compound No. | Weight | Structural Formula | Formula | MPO | LPO | TPO |
| Propylthiouracil (PTU) | 170.23 | 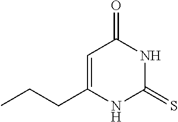 | C₇H₁₀N₂OS | 5.5 | n.s. | 0.119 |
| NE-2-6 | 344.38 | 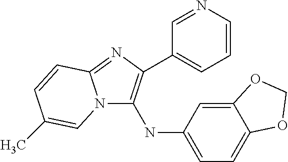 [SMILES: Cc1ccc2nc(-c3cccnc3)c(Nc3ccc4c(c3)OCO4)n2c1] | C20H16N4O2 | 6.45 | 0.89 | 0.013 |
| NE-2-7 | 373.42 | 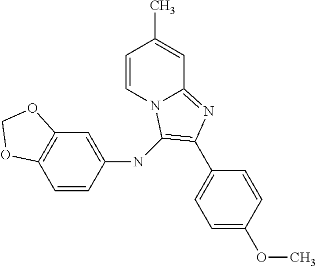 [SMILES: COc1ccc(-c2nc3cc(C)ccn3c2Nc2ccc3c(c2)OCO3)cc1] | C22H19N3O3 | 1.5 | 2.25 | 0.02 |
| NE-2-8 | 359.39 | 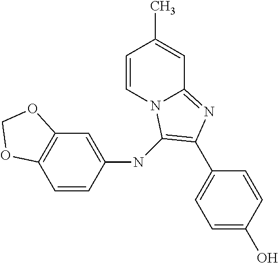 [SMILES: Cc1ccn2c(Nc3ccc4c(c3)OCO4)c(-c3ccc(O)cc3)nc2c1] | C21H17N3O3 | 0.77 | 1.05 | 0.016 |
| NE-2-9 | 413.50 | 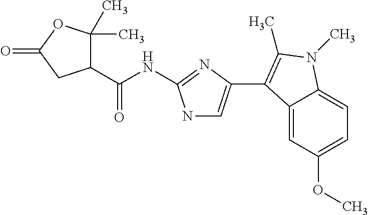 [SMILES: COc1ccc2c(c1)c(-c1c[nH]c(NC(=O)C3CC(=O)OC3(C)C)n1)c(C)n2C] | C21H23N3O4S | 1.83 | n.s. | 0.130 |

### Example 2

### Analysis for Effectiveness of Imidazopyridine Scaffold-Bearing Compounds as Therapeutics for Graves' Disease

### <2-1> Analysis of Inhibition Activity on Expression of Thyroid Peroxidase (TPO) and Thyroglobulin (TG)

To confirm whether the imidazopyridine scaffold-bearing compounds screened in Example 1 could be used as treatments for Graves' disease, SNU760 Human Thyroid cell lines were treated with various concentrations of each compound and then analyzed their ability to inhibit the expression of TPO (thyroid peroxidase) and TG (thyroglobulin).

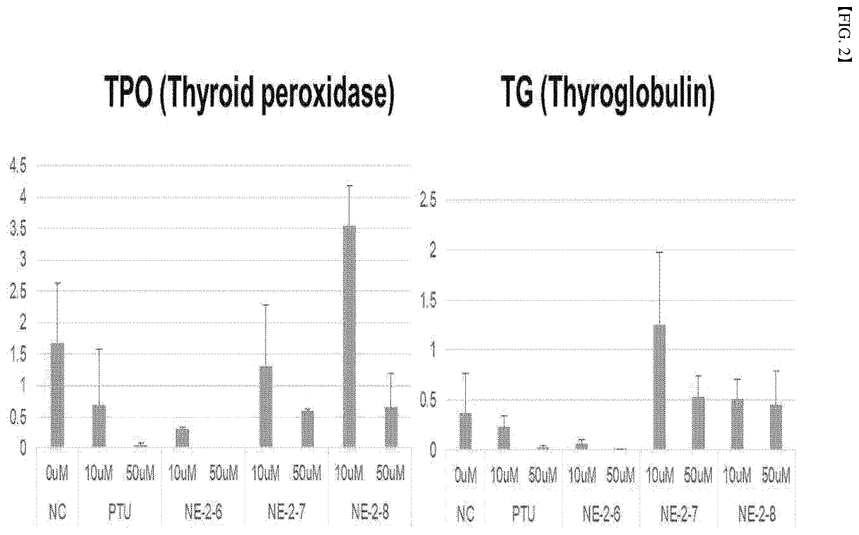

As shown in FIG. 2, the screened compounds effectively inhibited the expression of TPO and TG in a concentration-dependent manner. Notably, the compound NE-2-6 ((N-(2H-1,3-BENZODIOXOL-5-YL)-6-METHYL-2-(PYRIDIN-3-YL) IMIDAZO [1,2-A]PYRIDIN-3-AMINE)) at a concentration of 50 uM nearly completely inhibited the expression of TPO and TG by 100%, showing superior activity compared to the positive control drug, PTU.

### <2-2> Analysis of Cytotoxicity in Cell Lines

To ensure the safety of the Graves' disease candidate drugs used in experiment <2-1>, their cytotoxicity was tested by treating the SNU760 human thyroid cell lines with various concentrations of each compound.

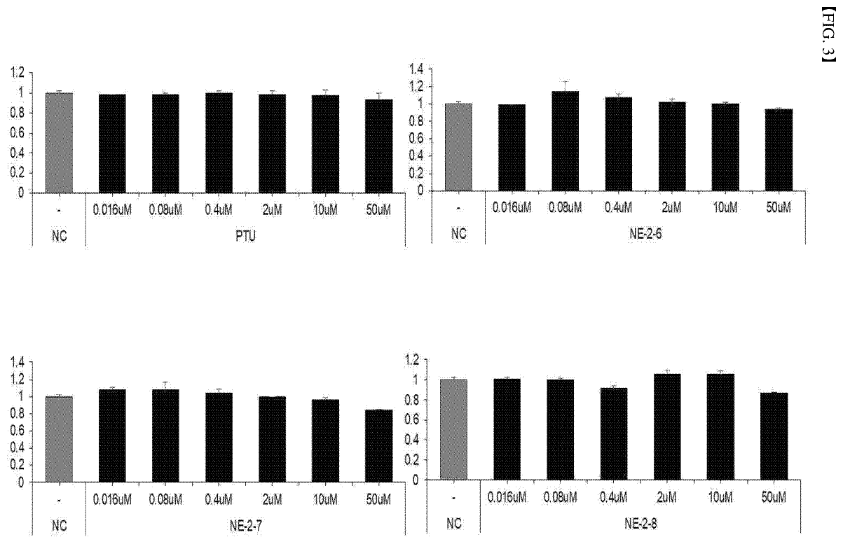

As depicted in FIG. 3, the compounds did not induce cytotoxicity in the cells. Therefore, it can be concluded that the imidazopyridine scaffold-bearing compounds screened for the treatment of Graves' disease are safe compounds with no cytotoxic effects.

### <2-3> Analysis of Toxicity in Mouse Animal Model

Furthermore, to verify whether the Graves' disease candidate drugs screened in the present disclosure are safe and non-toxic in actual animal models, the compound NE-2-7 was administered at a dose of 10 mg/kg to ICR mice and the changes in body weight were analyzed over time.

As illustrated in FIG. 4, the administration of the imidazopyridine scaffold-bearing compound did not induce significant changes in body weight, confirming that the screened compounds do not cause systemic toxicity. This finding supports the safety of using these compounds in potential clinical applications for treating Graves' disease.

### Example 3

### Analysis of Treatment Effect of Imidazopyridine Scaffold-Bearing Compounds in Graves' Disease-Induced Mice

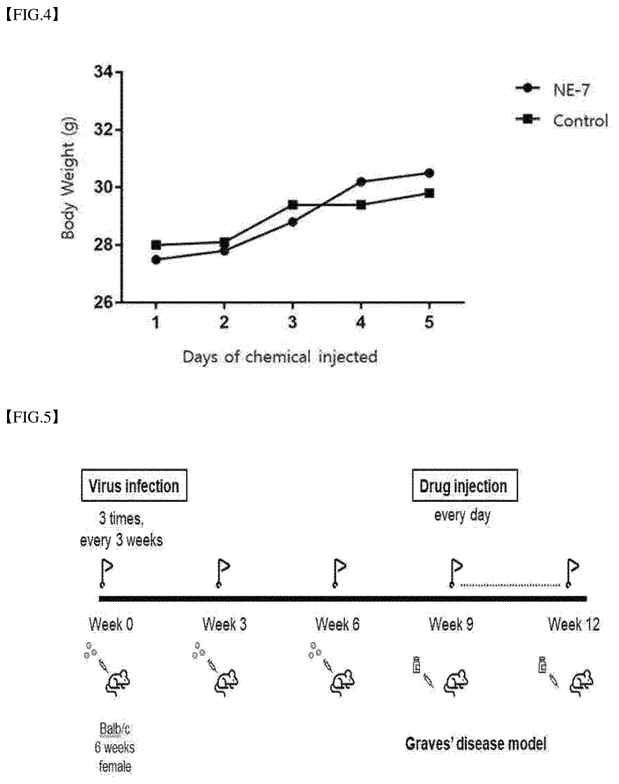

This experiment outlined the production of Graves' disease-induced mice and the administration process of the candidate drugs screened in the present disclosure, as depicted in FIG. 5.

### <3-1> Construction of Graves' Disease-Induced Mouse Model

To create a mouse model with induced Graves' disease, 6-week-old female BALB/c mice were used. The thyroid stimulating hormone receptor (TSHR) expressing adenovirus, Ad-TSHR, was purchased from VectorBuilder (Chicago, USA, www.vectorbuilder.com). A diluted solution of Ad-TSHR (3.02×10¹² VP/ml) was prepared, and 40 μl (10¹⁰ VP/40 μl) of the virus was injected into the thigh muscle of the mice. The virus was injected three times at three-week intervals, totaling nine weeks of injections to induce overexpression of TSHR and thereby cause Graves' disease.

Additionally, experiments were conducted to confirm whether Graves' disease was induced in the mice using the aforementioned method. After the injections of Ad-TSHR virus, thyroid tissues were extracted from the mice in the ninth week, and the transparency and cell size of the extracted thyroid tissues were analyzed. Normal mice that did not receive the Ad-TSHR virus injection served as the control group.

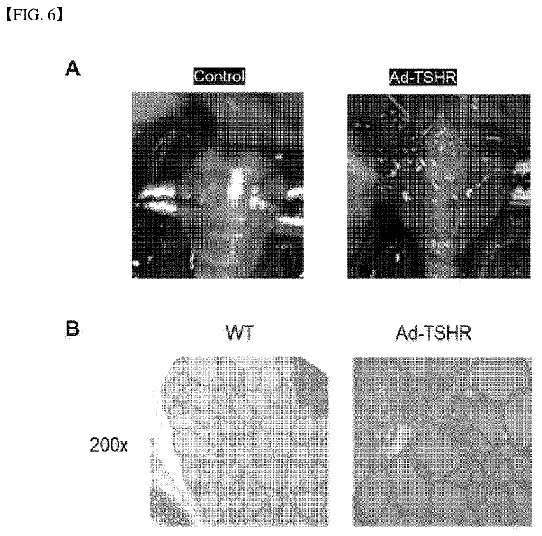

The analysis showed that, compared to the control group, the thyroid tissues of the mice injected with the Ad-TSHR virus had turned a darker red color (FIG. 6A), and the size of the thyroid tissue cells was abnormally larger, compared to the control, as shown in FIG. 6B (FIG. 6B).

These results confirmed that the mice developed Graves' disease using the described method. The disease-induced mouse model was then used to evaluate the therapeutic effects of the candidate drugs screened in the present disclosure in subsequent experiments

### <3-2> Verification of Therapeutic Effect of Imidazopyridine Scaffold-Bearing Compounds for Treating Graves' Disease

In the Graves' disease-induced mouse model established in <3-1>, the therapeutic effectiveness of the imidazopyridine scaffold-bearing compounds screened in the present disclosure was evaluated. To this end, a test group of the Graves' disease-induced mice was treated with the candidate drug NE2-6, identified through screening, at a daily dose of 10 mg/kg for three weeks. PTU was used as a positive control for comparison. The administration details of the candidate drugs are shown in Table 4 below.

TABLE 4

| | Ad-TSHR virus | Drug treatment on diseased | |
|---|---|---|---|
| | injection | mice (PTU, NE2-6) | |
| Injection site | Thigh | Abdominal cavity | |
| Solvent | PBS | Corn oil + DMSO | |
| Concentration | 10¹⁰ | 10 | mg/kg |
| Volume | 40 μl | 100 | μl |

| | | |
|---|---|---|
| Frequency | Every three weeks, | For three weeks, every day |
| | three times | |

The groups included: a normal control group (Control) that did not receive any treatment; a group that was injected with the Ad-TSHR virus to induce Graves' disease (Ad-TSHR); a positive control group that was injected with the Ad-TSHR virus followed by PTU medication (Ad-TSHR+PTU); and an experimental group that was injected with the Ad-TSHR virus followed by the NE2-6 compound from this invention (Ad-TSHR+NE2-6). After extracting the thyroid glands from each mouse, the sizes of the thyroids were visually compared, and the concentration of thyroxine (T4) in the blood was analyzed.

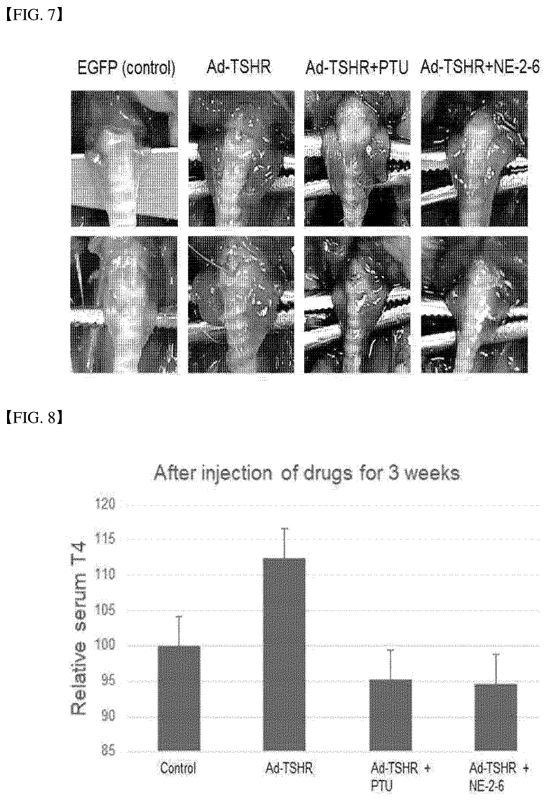

Sizes of the thyroid glands extracted from each mouse were measured with the eye and the results are depicted in FIG. 7. As understood from the data of FIG. 7, the group injected with the Ad-TSHR virus exhibited a significant increase in thyroid size due to the induced disease compared to the normal control group. In contrast, both the PTU treatment group and the group treated with the NE2-6 compound showed effectively reduced thyroid sizes compared to the Ad-TSHR group. Notably, the thyroid size in the NE2-6 treated group was nearly restored to normal levels.

After administering drugs for three weeks to mice in which Graves' disease was induced by the Ad-TSHR virus over nine weeks, the serum was isolated to measure the concentration of thyroxine (T4). As shown in FIG. 8, while the group injected with the Ad-TSHR virus showed a significant increase in thyroxine (T4) levels compared to the normal control group, both the PTU-treated group and the group treated with the NE2-6 compound demonstrated a significant reduction in the elevated levels of thyroxine (T4). Notably, the reduction in thyroxine (T4) levels was more effective in the NE2-6 treated group compared to the group treated with PTU, the conventionally known drug.

These results indicate that the imidazopyridine scaffold-bearing compound NE2-6, screened in the present disclosure, can reduce the activity and expression of MPO (myeloperoxidase), TPO (thyroid peroxidase), and TG (thyroglobulin). It can also decrease the abnormally enlarged thyroid size and reduce the concentration of thyroxine (T4) in the serum of mice induced with Graves' disease. Furthermore, it was confirmed that these compounds do not cause toxicity or adverse effects in the body, highlighting their safety. Conclusively, the imidazopyridine compounds identified and screened in the present disclosure have potential as new therapeutics for the treatment of Graves' disease.

While this present general inventive concept has been particularly illustrated and described with reference to exemplary embodiments thereof, it will be appreciated by those of ordinary skill in the art that changes may be made in these exemplary embodiments without departing from the principles and spirit of the present general inventive concept, the scope of which is defined in the appended claims and their equivalents. The exemplary embodiments should be considered in descriptive sense only and not for purposes of limitation. Therefore, the scope of the present general inventive concept is defined not by the detailed description of the present general inventive concept but by the appended claims, and all differences within the scope will be construed as being included in the present general inventive concept.

1. A pharmaceutical composition for prevention or treatment of Graves' disease, the composition comprising an imidazopyridine scaffold-bearing compound as an active ingredient.

2. The pharmaceutical composition of claim 1, wherein the compound is any one selected from the group consisting of the following Structural Formulas 1 to 3:

| No. | Chemical Structural Formula |
|---|---|
| 1 | 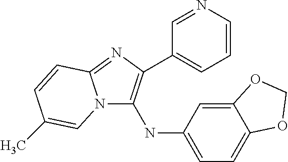 [SMILES: Cc1ccc2nc(-c3cccnc3)c(Nc3ccc4c(c3)OCO4)n2c1] |
| 2 | 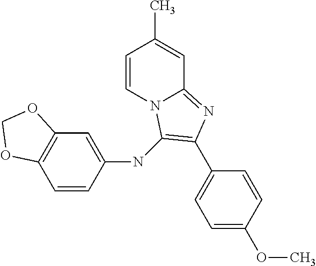 [SMILES: COc1ccc(-c2nc3cc(C)ccn3c2Nc2ccc3c(c2)OCO3)cc1] |
| 3 | 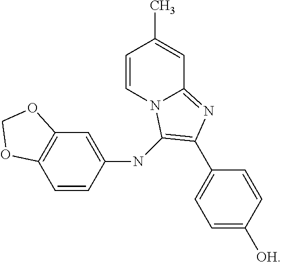 [SMILES: Cc1ccn2c(Nc3ccc4c(c3)OCO4)c(-c3ccc(O)cc3)nc2c1] |

3. The pharmaceutical composition of claim 1, wherein the compound exhibits activities of:
inhibiting the expression of thyroid peroxidase (TPO) and thyroglobulin (TG);
reducing the size of abnormally enlarged thyroid tissues and cells; and
lowering the serum concentration of the thyroid hormone thyroxine.

4. A health functional food, comprising an imidazopyridine scaffold-bearing compound as an active ingredient for prevention or alleviation of Graves' disease.

5. The health functional food composition of claim 4, wherein the compound is any one selected from the group consisting of the following Structural Formulas 1 to 3:

| No. | Chemical Structural Formula |
|---|---|
| 1 | 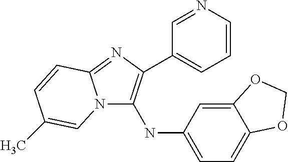 [SMILES: Cc1ccc2nc(-c3cccnc3)c(Nc3ccc4c(c3)OCO4)n2c1] |
| 2 | 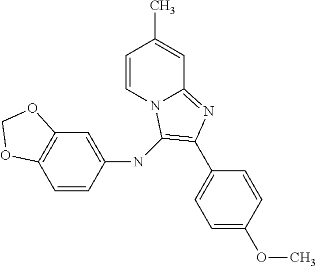 [SMILES: COc1ccc(-c2nc3cc(C)ccn3c2Nc2ccc3c(c2)OCO3)cc1] |
| 3 | 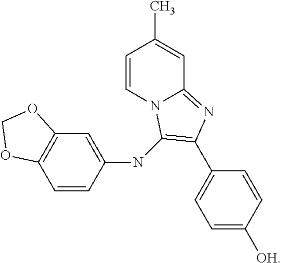 [SMILES: Cc1ccn2c(Nc3ccc4c(c3)OCO4)c(-c3ccc(O)cc3)nc2c1] |

6. The pharmaceutical composition of claim 4, wherein the compound exhibits activities of:
inhibiting the expression of thyroid peroxidase (TPO) and thyroglobulin (TG);
reducing the size of abnormally enlarged thyroid tissues and cells; and
lowering the serum concentration of the thyroid hormone thyroxine.

7. A method for treating Graves' disease, the method comprising a step of administering an imidazopyridine scaffold-bearing compound to an animal afflicted with Graves' disease.

8. The method of claim 7, wherein the compound is any one selected from the group consisting of the following Structural Formulas 1 to 3:

| No. | Chemical Structural Formula |
|---|---|
| 1 | 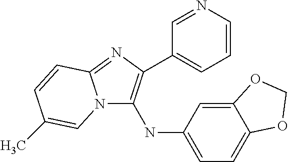 [SMILES: Cc1ccc2nc(-c3cccnc3)c(Nc3ccc4c(c3)OCO4)n2c1] |
| 2 | 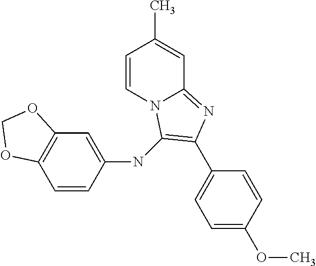 [SMILES: COc1ccc(-c2nc3cc(C)ccn3c2Nc2ccc3c(c2)OCO3)cc1] |
| 3 | 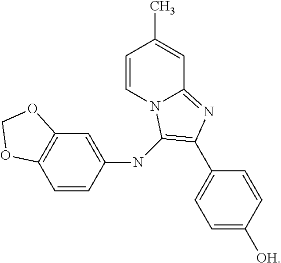 [SMILES: Cc1ccn2c(Nc3ccc4c(c3)OCO4)c(-c3ccc(O)cc3)nc2c1] |
